(19) United States
(12) Patent Application Publication
(10) Pub. No.: US 2011/0136954 A1
(43) Pub. Date: Jun. 9, 2011

(54) BIOSOURCED ACROLEIN POLYMER, METHOD FOR OBTAINING SAME, AND USES THEREOF

(75) Inventor: Jean-Luc Dubois, Millery (FR)

(73) Assignee: ARKEMA FRANCE, Colombes (FR)

(21) Appl. No.: 12/999,879

(22) PCT Filed: Jun. 19, 2009
(86) PCT No.: PCT/FR2009/051171

§ 371 Date: Feb. 24, 2011

(30) Foreign Application Priority Data
Jun. 19, 2008 (FR) 0854057

(51) Int. Cl. C08L 29/00; C08F 16/34; C08F 2/00; C08F 8/28; C08K 3/26; C08K 5/05

(52) U.S. Cl. 524/379; 526/315; 526/75; 525/384; 524/424; 524/543

(57) ABSTRACT

The present invention relates to an acrolein polymer containing a carbon biosourced according to the ASTM D6866-06 standard, and to the preparation method thereof including a first step for dehydrating glycerol from a glycerol aqueous solution in the presence of an acid catalyst, followed by a polymerization of the obtained substance in the presence of an ionic catalyst or a free radical initiator. The polymer according to the invention has numerous uses in the fields of animal health, antibiotics, disinfection and sterilization, cosmetics, drilling sludge, paints and inks, and the paper and textile industries, and responds to certain sustainable development concerns.

The present invention relates to an acrylic polymer derived from renewable starting materials, to the process for preparing it and to its uses. In particular, the present invention relates to a polymer derived from acrolein, containing biosourced carbon, to the process for preparing it from glycerol, and to its uses.

Acrolein (also known as 2-propenal) finds many applications in industry, especially by virtue of the presence of its two reactive functions, vinyl and aldehyde, which can react individually or together. It is the starting product for a large number of industrial chemical reactions, in particular for the manufacture of methionine, a synthetic protein used as an animal food supplement, acrylic acid, the importance of the derivatives of which is appreciated, and glutaraldehyde, which has many uses as a biocidal agent or disinfectant. Acrolein is also used as a biocidal agent in manufacturing lines and industrial waters.

Acrolein is a highly reactive compound that readily polymerizes in the presence of bases, amines, strong acids or peroxides to give polymers generally known as polyacroleins.

Various manufacturing processes, based on the use of different catalysts, make it possible to produce polyacrolein polymers, and may lead to different polymer characteristics, especially in terms of solubility in organic solvents or in aqueous medium, molecular mass range, content of carbonyl functions, etc. Examples of acrolein polymerization catalysts that may be mentioned include basic compounds such as alkali metal hydroxides, aliphatic amines, sodium carbonate, aqueous ammonia, hydrazine, piperidine, free-radical catalysts such as peroxides, for instance benzoyl peroxide, tert-butyl hydroxyperoxide, tert-butyl perbenzoate, tert-butyl peracetate, alkali metal persulfates, percarbonates or perborates, azo compounds, redox systems comprising a reducing agent such as sulfites or bisufites, etc. The polymerization may be performed in aqueous medium or in the presence of a solvent such as light alcohols, ethers such as THF or dioxane, aliphatic or aromatic hydrocarbons such as hexane, benzene, toluene or cyclohexane, chlorinated hydrocarbons such as methylene chloride, chlorobenzene, etc. Reference may be made especially to the following documents that describe various processes for obtaining acrolein-based polymers: CA 696 969; FR 1 312 166; FR 1 299 254; U.S. Pat. No. 1,051,858; U.S. Pat. No. 2,657,192; U.S. Pat. No. 3,635,898; GB 1 051 858; WO 88/04671; EP 667 358.

Acrolein may also be subjected to a copolymerization reaction with other vinyl monomers, such as methyl methacrylate, styrene, acrylic esters or vinyl acetate, thus leading to a wide range of products. For example, mention may be made of document FR 1 299 254 which describes the preparation of acrolein/acrylonitrile and acrolein/methyl methacrylate copolymers. Document FR 1 312 166 describes copolymers of acrolein and of β-substituted acroleins, such as aryl, aralkyl, alkyl and alkaryl acroleins, or of other monomers containing an ethylenic group.

After their synthesis, the acrolein polymers may be subjected to various chemical treatments in order to modify their characteristics or their application properties. For example, polymers that are insoluble in water or in standard solvents such as benzene, toluene or acetone may be converted into a soluble form by treatment using various materials such as sulfur dioxide, sodium sulfite, mercaptans, alcohols, etc.

Mention may also be made of flame-retardant polymers, described in document U.S. Pat. No. 3,183,214, obtained from the reaction of diphosphites with the aldehyde functions of polyacrolein polymers. In documents WO 01/60874 and WO 2005/044874, the acrolein-based polymers are subjected to a heat treatment at between 40 and 150° C. in the presence of water and/or of an alcohol or a polyol, so as to improve their stability and their antimicrobial properties.

Acrolein-based polymers find wide openings especially in the fields of animal health, antibiotics, disinfection and sterilization, cosmetics, drilling muds, paints and inks, the paper and textile industry, etc. In particular, the acrolein polymers sold under the brand name Chemyde are antibacterial products used as alternatives to antibiotics for treating bacterial infections in animal health.

The acrolein production process most commonly used is based on the gas-phase catalytic oxidation reaction of propylene with atmospheric oxygen. The production of the acrolein polymers, which is directly linked to that of acrolein, is thus greatly dependent on the propylene starting material obtained by vapor cracking or catalytic cracking of petroleum fractions. This starting material, of fossil origin, contributes toward increasing the greenhouse effect. In addition, this process uses petroleum, the natural deposits of which are rapidly running out; its extraction is increasingly difficult (wells of greater depth), requiring heavy and expensive equipment, which needs to withstand high temperatures (400-500° C.). Given the decrease in worldwide petroleum reserves, the source of these starting materials will gradually run out.

Starting materials derived from biomass are of renewable source, or biosourced, and have a reduced impact on the environment. The term “biomass” means a naturally produced starting material of plant or animal origin. This plant matter is characterized in that the plant, for its growth, has consumed atmospheric CO₂ while producing oxygen. Animals, for their growth, have consumed this plant starting material and have thus assimilated the carbon derived from atmospheric CO₂.

Biomass-based starting materials do not require all the extraction and refining steps, which are very costly in energy terms, of petroleum products. The production of CO₂ is reduced, and as such they contribute less toward climatic heating and satisfy certain sustainable development concerns.

Moreover, in fields such as cosmetics or animal health, consumers are increasingly drawn toward products of plant origin, which have the reputation of being safer in terms of ecological and biological ethics.

It thus appears necessary to have available acrolein-based polymers that are not dependent on starting material of fossil origin, and consequently a process for synthesizing acrolein-based polymers using biosourced starting materials.

It is known practice to synthesize acrolein by dehydration of glycerol. Glycerol (also known as glycerin when it is in the form of an aqueous solution) is especially derived from the methanolysis of plant and animal oils at the same time as the methyl esters, which themselves are used especially as fuels or combustibles in diesel and domestic fuel oil. It is a natural product, available in large amount, and may be stored and transported without difficulty. It has the advantage of being a renewable starting material that satisfies the criteria associated with the new concept of “green chemistry”.

Many recent studies have been devoted to the upgrading of glycerol and especially to the preparation of acrolein. The process that is the subject of these studies uses a glycerol dehydration reaction according to the following reactions:

CH₂OH—CHOH—CH₂OH→CH₂OH—CH₂—CHO+H₂O [symbol] CH₂═CH—CHO+2H₂O

which make it possible to obtain acrolein.

This reaction is an equilibrated reaction; as a general rule, the hydration reaction is favored at low temperatures, and dehydration is favored at high temperatures. To obtain acrolein, a sufficient temperature must thus be used, and/or a partial vacuum must be used to shift the reaction. The reaction may be performed in the liquid phase or in the gas phase. This type of reaction is known to be catalyzed with acids. Reference may be made, for example, to documents FR 695 931; U.S. Pat. No. 2,558,520; WO 99/05085; U.S. Pat. No. 5,387,720; WO 06/087 083 and WO 06/087 084, which describe various conditions for performing the glycerol dehydration reaction to produce acrolein.

The acrolein obtained from glycerol is particularly advantageous for the synthesis of acrolein-based polymers, which may then be said to be “obtained from biomass” or “biosourced”. In process terms in particular, most of the impurities present in the acrolein derived from glycerol cannot be polymerized in the process for obtaining the polymer. They will then be removed at the same time as the excess acrolein in the subsequent treatment directed toward freeing the polymer of the residual monomer. Acrolein derived from the dehydration of glycerol also contains less acrylic acid and acetic acid than acrolein derived from the oxidation of propylene. However, it contains more propanaldehyde and acetaldehyde.

A subject of the present invention is thus, firstly, an acrolein-based polymer in which at least some of its carbons are biosourced.

A biosource is a natural animal or plant source, the stock of which can be reconstituted over a short period on the human timescale. It is in particular necessary for this stock to be able to be renewed as quickly as it is consumed.

In contrast with materials derived from fossil materials, biosourced starting materials contain ¹⁴C in the same proportions as atmospheric CO₂. All the carbon samples taken from living organisms (animals or plants) are in fact a mixture of three isotopes: ¹²C (representing about 98.892%), ¹³C (about 1.108%) and ¹⁴C (traces: 1.2×10⁻¹⁰%). The ¹⁴C/¹²C ratio of living tissues is identical to that of the atmosphere. In the environment, ¹⁴C exists in two predominant forms: in mineral form, i.e. carbon dioxide (CO₂), and in organic form, i.e. carbon incorporated into organic molecules.

In a living organism, the ¹⁴C/¹²C ratio is kept constant by the metabolism since the carbon is continually exchanged with the environment. Since the proportion of ¹⁴C is constant in the atmosphere, this is likewise the case in the organism, as long as it is alive, since it absorbs this ¹⁴C as it absorbs the ¹²C. The mean ¹⁴C/¹²C ratio is equal to 1.2×10⁻¹². Carbon-14 is derived from the bombardment of atmospheric nitrogen (14), and becomes oxidized spontaneously with atmospheric oxygen to give CO₂. In our human history, the content of ¹⁴CO₂ increased after atmospheric nuclear explosions, and has since not ceased to decrease after the stoppage of these tests.

¹²C is stable, i.e. the number of ¹²C atoms in a given sample is constant over time. ¹⁴C is, itself, radioactive (each gram of carbon of a living being contains enough ¹⁴C isotopes to give 13.6 disintegrations per minute) and the number of such atoms in a sample decreases over time (t) according to the law:

n=nₒexp(−at),

in which:
nₒ is the number of ¹⁴C at the origin (on the death of the creature, animal or plant),
n is the number of ¹⁴C atoms remaining after time t,
a is the disintegration constant (or radioactive constant); it is linked to the half-life.

The half-life (or period) is the time after which any number of radioactive nuclei or of unstable particles of a given species is reduced by half by disintegration; the half-life T_{1/2} is linked to the disintegration constant a by the formula aT_{1/2}=In 2. The half-life of ¹⁴C is 5730 years. In 50 000 years, the content of ¹⁴C is less than 0.2% of the initial content and thus becomes difficult to detect. Petroleum products, or natural gas or coal, therefore contain no ¹⁴C.

Given the half-life (T_{1/2}) of ¹⁴C, the content of ¹⁴C is substantially constant from the extraction of the biosourced starting materials to the manufacture of the “biomaterials” derived from these starting materials and even up to the end of their use.

The ¹⁴C content of a “biomaterial” may be deduced from measurements taken, for example, according to the following techniques:
by liquid scintillation spectrometry: this method consists in counting the “beta” particles derived from the disintegration of C. The beta radiation derived from a sample of known mass (known number of carbon atoms) over a certain time is measured. This “radioactivity” is proportional to the number of ¹⁴C atoms, which may thus be determined. The ¹⁴C present in the sample emits β rays, which, on contact with the scintillant liquid, give rise to photons. These photons have different energies (between 0 and 156 Key) and form what is known as a ¹⁴C spectrum. According to two variants of this method, the analysis relates either to the CO₂ produced beforehand by combustion of the carbon sample in a suitable absorbent solution, or to the benzene after prior conversion of the carbon-based sample into benzene;
by mass spectrometry: the sample is reduced to graphite or to gaseous CO₂, and analyzed in a mass spectrometer. This technique uses an accelerator and a mass spectrometer to separate the ¹⁴C ions from the ¹²C ions and thus to determine the ratio of the two isotopes.

These methods for measuring the ¹⁴C content of materials are described precisely in standards ASTM D 6866 (especially D6866-06) and in standards ASTM D 7026 (especially 7026-04). These methods compare the data measured on the analyzed sample with the data of a reference sample of 100% biosourced origin (for which the ¹⁴C/¹²C is 1.2×10⁻¹²), to give a relative percentage of biosourced carbon in the sample. The ¹⁴C/¹²C ratio of the sample may thus be deduced therefrom.

The measuring method preferentially used is mass spectrometry described in standard ASTM D6866-06 (“accelerator mass spectroscopy”).

One subject of the present invention is thus an acrolein-based polymer, characterized in that it has a mass content of ¹⁴C such that the ¹⁴C/¹²C ratio is between 0.2×10⁻¹² and 1.2×10⁻¹² according to standard ASTM D 6866, and preferably the ¹⁴C/¹²C ratio is between 0.6×10⁻¹² and 1.2×10⁻¹² and more particularly between 0.8×10⁻¹² and 1.2×10⁻¹².

In one preferred embodiment, the acrolein-based polymer of the invention is such that the ratio ¹⁴C/¹²C is equal to 1.2×10⁻¹², i.e. it contains 100% biosourced carbon.

The term “acrolein-based polymer” means a polymer containing at least units of the type:

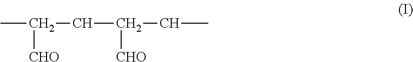

which may be in the hydrated, hemiacetal or acetal form, according to the representations given in FIGS. 1 and 2 of patent application WO 88/04671, or patent application WO 96/38186, which are incorporated into the present description. These units may result from the homopolymerization of acrolein or from the copolymerization of acrolein with at least one polymerizable comonomer, the polymerization being performed starting with the aldehyde function and/or the vinyl function of acrolein.

The units (I) comprising aldehyde functions may be in hydrated diol form, in hemiacetal or acetal form resulting from the condensation of the diol form with the aldehyde or diol form, in tetrahydropyran or polytetrahydropyran form formed from the condensation of the diol form, or in a form resulting from an aldol-Michael condensation.

Advantageously, the proportion of aldehyde functions corresponding to the unit of formula (I) is less than 20 mol % and preferably between 5 and 15 mold in the polymer.

Comonomers that may be mentioned include β-substituted acroleins such as aryl, arylalkyl, alkyl and alkylaryl acroleins, for instance β-ethylacrolein, β-phenyl-acrolein, β-butylacrolein, β-octylacrolein, aldehyde derivatives of acrolein, for instance acrolein diallyl acetals, monomers containing at least one ethylenic group, preferably a group CH₂—CH—, such as butadiene, isoprene, methylpentadiene, cyclopentadiene, chloro-prene, ethylene, propylene, butylene, octene, vinyl acetate, vinyl propionate, vinylpyridine, vinyl-naphthalene, styrene, vinylcyclohexane, vinyl chloride, vinylidene chloride, acrylic derivatives such as acrylic acid, methacrylic acid, acrylonitrile, meth-acrylonitrile, (meth)acrylic esters such as methyl methacrylate, ethyl acrylate, butyl acrylate, allylic compounds, such as allyl acetate, allyl alcohol, allyl-amine, diallyl succinate, etc.

A preferred comonomer is acrylic acid.

In one preferred form of the invention, the comonomers are derived from biosourced starting materials, for example the acrylic acid may be obtained from glycerol and may contain biosourced carbon.

The comonomers are generally used in an amount from about 0% to 40% by weight, preferably from 0 to 25% by weight and more particularly from 0 to 10% by weight relative to the weight of the polymer.

The polymers according to the invention have a molecular mass that may vary within a wide range. The preferred polymers have a weight-average molecular mass of at least 1000, preferably of at least 2000 and more particularly between 2000 and 10 000.

Preferred polymers are those with a high content of aldehyde groups present in hydrated form.

Other preferred polymers are those, containing carboxylic acid functions resulting from the partial autoxidation of the aldehyde units. Preferably, the content of carboxylic units is between 0.1 and 5 mol per kilogram of polymer.

A subject of the present invention is also a process for preparing an acrolein-based polymer comprising units of the type:

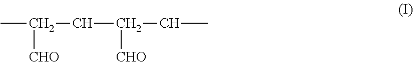

which may be in hydrated, hemiacetal or acetal form, comprising the following steps:
(a) the dehydration of glycerol starting with an aqueous solution of glycerol in the presence of an acid catalyst, to produce a mixture of acrolein and water in a first reactor;
(b) the optional purification of the product obtained in step (a);
(c) the polymerization in a second reactor of the acrolein produced in (a) or (b);
(d) the recovery of the polymer produced in (c), optionally followed by steps of washing, drying and/or molecular distillation and activation in air.

The first step a) of dehydration of the glycerol is performed in the gas phase in a reactor in the presence of a catalyst at a temperature ranging from 150° C. to 500° C. and preferably between 250° C. and 350° C., and a pressure of between 10⁵ and 5×10⁵ Pa.

The reactor used may function as a fixed bed, as a fluidized bed or as a circulating fluidized bed, or in a modular configuration (plates or baskets) as described in documents EP 995 491, EP 1 147 807 or US 2005/0 020 851, in the presence of acidic solid catalysts.

The catalysts that are suitable for use are homogeneous or multiphase materials, which are insoluble in the reaction medium that has a Hammett acidity, noted as H₀, of less than +2 as indicated in U.S. Pat. No. 5,387,720 which refers to the article by K. Tanabe et al. in “Studies in Surface Science and Catalysis”, Vol. 51, 1989, chapters 1 and 2; the Hammett acidity is determined by amine titration using indicators or by adsorption of a base in the gaseous phase. The catalysts corresponding to the acidity criterion H₀ of less than +2 may be chosen from natural or synthetic siliceous materials or acidic zeolites; mineral supports, such as oxides, covered with inorganic acids, mono-, di-, tri- or polyacids; oxides or mixed oxides, or alternatively heteropolyacids.

Advantageously, strongly acidic solid catalysts with a Hammett acidity H₀ of between −9 and −18 are used.

These catalysts may generally be constituted by a heteropolyacid salt in which protons of said heteropolyacid are exchanged with at least one cation chosen from the elements belonging to groups I to XVI of the Periodic Table of the Elements, these heteropolyacid salts containing at least one element chosen from the group comprising W, Mo and V.

Among the mixed oxides that may also be mentioned are those based on iron and phosphorus and those based on cesium, phosphorus and tungsten.

Advantageously, the catalysts are chosen from zeolites, Nafion® composites (based on sulfonic acid of fluoro-polymers), chlorinated aluminas, phosphotungstic and/or silicotungstic acids and acid salts, and various solids of metal oxide type such as tantalum oxide Ta₂O₅, niobium oxide Nb₂O₅, alumina Al₂O₃, titanium oxide TiO₂, zirconia ZrO₂, tin oxide SnO₂, silica SiO₂ or silico-aluminate SiO₂—Al₂O₃, impregnated with acidic functions such as borate BO₃, sulfate SO₄, tungstate WO₃, phosphate PO₄, silicate SiO₂ or molybdate MoO₃. According to the literature data, these catalysts all have a Hammett acidity H₀ of less than +2.

Preferred catalysts are sulfated zirconias, phosphated zirconias, tungstated zirconias, siliceous zirconias, sulfated titanium or tin oxides, or phosphated, phosphotungstated or silicotungstated aluminas or silicas.

In one particular embodiment of the invention, the glycerol dehydration reaction is performed in the presence of molecular oxygen, as is described in document WO 06/087 083.

Glycerol is available in concentrated form, but also in more economical aqueous solutions. Advantageously, an aqueous glycerol solution with a concentration of between 10% and 50% by weight and preferably between 15% and 30% by weight is used in the reactor. The concentration should not be too high, so as to avoid parasite reactions such as the formation of glycerol ethers or reactions between the acrolein or the acrylic acid produced and glycerol. Moreover, the glycerol solution should not be too dilute on account of the energy cost incurred in evaporating the aqueous glycerol solution. In all cases, the concentration of the glycerol solution may be adjusted by recycling the water produced by the reaction. In order to reduce the glycerol transportation and storage costs, the reactor may be fed with concentrated solution of 40% to 100% by weight, the dilution to the optimum content being performed by recycling some of the water vapor produced by the reaction and some of the dilution water. Similarly, the recovery of heat at the reactor outlet may also allow the glycerol solution feeding the reactor to be vaporized.

Glycerol derived from the methanolysis of plant oils in basic medium may contain certain impurities such as sodium or potassium chloride or sulfate, non-glyceric organic matter, and methanol. A prior purification treatment of the glycerol may be envisioned, for example by ion exchange.

In one particular embodiment of the process according to the invention, the aqueous glycerol solution is vaporized in a fluidized bed containing an inert solid such as sand, glass or quartz powder, silicon carbide, or a solid with a low specific surface area, maintained at a temperature of between 220 and 350° C., making it possible simultaneously to remove the impurities present in the glycerol solution or generated in the course of the evaporation in this solution.

The dehydration reaction of the glycerol to acrolein is generally accompanied by side reactions leading to the formation of byproducts, such as hydroxypropanone, propanaldehyde, acetaldehyde, acetone, phenol, acrylic acid, adducts of acrolein with glycerol, products of polycondensation of glycerol, and cyclic glycerol ethers. Acrolein derived from the dehydration of glycerol generally contains less acrylic acid and acetic acid than acrolein derived from the oxidation of propylene. However, it contains more propanaldehyde and acetaldehyde.

The product obtained in step (a) of the process according to the invention contains not only the acrolein produced and the abovementioned byproducts, but also a large amount of water, originating firstly from the glycerol solution, and secondly from the water produced by the dehydration reaction.

It may be advantageous to purify the product obtained in step (a) to facilitate and optimize the polymerization step, in particular to remove the majority of the water present, and/or to remove the light aldehydes (formaldehyde, acetaldehyde, etc.) whose presence may be harmful with respect to the polymerization catalyst.

Advantageously, a step is performed that consists in at least partly condensing the water and heavy byproducts present in the stream derived from the first dehydration step, before performing step (c) of acrolein polymerization. This step may be performed with a condensation unit that may be an adsorption column optionally coupled to an evaporator, a heat exchanger, a condenser, a deflegmator, and also any apparatus that is well known to those skilled in the art, for performing partial condensation of an aqueous stream. Generally, after this step, a stream of acrolein containing about 5% water is obtained.

Other treatments directed toward purifying the acrolein thus produced, freed of the majority of the water and heavy byproducts, may be performed to obtain an acrolein of “polymer grade” quality. Mention may be made of distillation, liquid-liquid extraction and membrane separation.

In one preferred embodiment, a first distillation of the acrolein stream is performed so as to remove the acetaldehyde that may be present up to contents of about 10% to 15%. This distillation may be performed continuously at atmospheric pressure, using a side-fed distillation column stabilized with a stream of a polymerization inhibitor, for instance hydroquinone or any other polymerization inhibitor conventionally used for stabilizing acrolein. This distillation is performed so as to remove more than 90% of the acetaldehyde, preferably to obtain an acrolein stream comprising less than 5% acetaldehyde and preferably less than 1% acetaldehyde.

A second distillation may be performed to achieve this level of purity.

The acrolein obtained after step (b) generally has a purity of greater than 70 mol % and preferably greater than 90 mol % (excluding the water present), more particularly a purity of greater than 95%. It may also contain water to a content ranging from 3% to 5%.

According to one particular embodiment of the invention, after step (b), the acrolein contains acrylic acid, which will consequently be copolymerized with the acrolein in step (c).

Advantageously, a polymerization inhibitor may be added to the acrolein, for instance hydroquinone, to a content that may range from 0.01% to 0.5% by weight so as to limit the autodimerization of acrolein before performing the polymerization reaction of step (c).

The acrolein polymerization reaction may be performed in the presence of an ionic catalyst such as a base, for instance an alkali metal hydroxide NaOH or LiOH, or in the presence of a free-radical initiator such as peroxides, for instance benzoyl peroxide, tert-butyl hydroxyperoxide, tert-butyl perbenzoate or tert-butyl peracetate, or azo derivatives.

Any other method known to those skilled in the art may be used.

The polymerization may be performed in aqueous medium or in an organic solvent such as an alcohol, for example methanol.

Preferably, the acrolein polymerization reaction is performed in aqueous solution in the presence of sodium hydroxide.

Advantageously, the polymerization reaction is performed in the presence of air and/or oxygen, with or without the presence of an inhibitor. As indicated in patent application WO 00/03723, the presence of air and/or oxygen makes it possible to obtain a product in crystalline form whose separation and drying are facilitated, and which also has improved inherent antimicrobial activity.

Advantageously, the acrolein polymerization reaction is performed at a temperature that may range from 0° C. to 100° C. and preferably from room temperature to 80° C., i.e. from 20° C. to 80° C.

According to one embodiment of the process according to the invention, the polymerization is performed in the second reactor in the presence of at least one comonomer that is polymerizable with acrolein.

Comonomers that may be mentioned include β-substituted acroleins such as aryl, arylalkyl, alkyl and alkylaryl acroleins, for instance β-ethylacrolein, β-phenyl-acrolein, β-butylacrolein, β-octylacrolein, aldehyde derivatives of acrolein, such as acrolein dialkyl acetals, monomers containing at least one ethylenic group, preferably a group CH₂═CH—, such as butadiene, isoprene, methylpentadiene, cyclopentadiene, chloro-prene, ethylene, propylene, butylene, octene, vinyl acetate, vinyl propionate, vinylpyridine, vinyl-naphthalene, styrene, vinylcyclohexane, vinyl chloride, vinylidene chloride, acrylic derivatives such as acrylic acid, methacrylic acid, acrylonitrile, meth-acrylonitrile, esters such as methyl methacrylate, ethyl acrylate, butyl acrylate, allylic compounds, such as allyl acetate, allyl alcohol, allylamine, diallyl succinate, etc.

A preferred comonomer is acrylic acid.

The comonomers are generally used in an amount of about 0% to 40% by weight, preferably from 0 to 25% by weight and more particularly from 0 to 10% by weight relative to the polymer.

The polymer produced in step (c) is recovered by filtration and/or centrifugation, and may be subjected to various treatments such as washing with water, drying, or milling to reduce the particle size. It may also be recovered by molecular distillation, scraped falling-film evaporation, evaporation on a spinning disk (spinning disk evaporator), or any other type of process for evaporating under vacuum the residual monomers and similarly the other impurities that were present with the acrolein and that were not able to polymerize, such as propanaldehyde and acetaldehyde. A combination of techniques described above may also be used.

According to one embodiment of the process according to the invention, an additional step (e) is performed, which consists in at least partially oxidizing the aldehyde functions of the polymer recovered in (d) in dry form, by heating said polymer to a temperature ranging from room temperature to 110° C. and preferably from 60° C. to 85° C., in the presence of air, for a time that may range from a few hours to a few days. Typically, this step may be performed using a temperature gradient ranging, for example, from 40° C. to 85° C., with intermediate stages lasting 2 hours to 24 hours.

According to one embodiment of the process according to the invention, an additional step (e) is performed, which consists in heating the polymer in the presence of water, optionally in the presence of a polyethylene glycol, a polyol or an alkanol, at a temperature ranging from 40° C. to 150° C. and preferably from 40° C. to 115° C., for a time ranging from 1 to 1400 hours and preferably from 10 to 60 hours, as described in document WO 01/60874.

According to one embodiment of the process according to the invention, an additional step (e) is performed, which consists in dissolving the polymer recovered in step (d), in an alcohol or a polyol, optionally in the presence of water, to form a solution with a pH of less than or equal to 7, in heating the solution thus obtained to form a product of reaction between said polymer and the alcohol or the polyol, as described in document WO 2005/044874. This step has the consequence of activating the antimicrobial properties of the polymer.

As examples of alcohols or polyols that may be used in the process according to the invention, mention may be made of polyalkylene glycols, such as polyethylene glycols, preferably those with a weight-average molecular mass ranging from 200 to 20 000, more particularly polyethylene glycols with a weight-average molecular mass ranging from 200 to 2000.

According to one embodiment of the process according to the invention, an additional step (e) is performed, which consists in placing the polymer in contact with a linear or branched C₁ to C₁₀ alcohol, as described in document FR 2 802 933.

According to one embodiment of the process according to the invention, an additional step (e) is performed, which consists in dissolving the polymer recovered in step (d), in a basic aqueous solution such as a sodium carbonate solution.

A subject of the present invention is also a composition comprising at least one acrolein-based polymer as described previously or obtained according to the process described previously.

The composition according to the invention may be in the form of a solution or in the form of an emulsion.

The emulsion according to the invention may comprise a polymeric oily phase, an aqueous phase, an emulsifier, and one or more stabilizers.

The composition may also comprise additives such as dispersants, pigments, biocidal agents, etc.

A subject of the present invention is also the use of the acrolein-based polymer as described previously or obtained according to the process described previously, in compositions to give them biocidal or disinfectant properties.

A subject of the present invention is also the use of the acrolein-based polymer as described previously or obtained according to the process described previously, for preparing medicaments intended for treating or preventing infections, food additives for cattle, dermatological compositions, or as a preserving agent or biocidal agent in applications in drilling muds, paints, plastics, inks, paper and textiles.

The examples that follow illustrate the present invention without, however, limiting its scope.

### EXAMPLES

### Example 1

### Preparation of Acrolein from Glycerol

A Pyrex reactor containing a bed of catalyst retained by a sinter is used. A catalyst such as the tungstated zirconia dehydration catalyst from Dailchi Kigenso KK, of reference Z1044 with a mass of about 6.6 g diluted with 7 ml of silicon carbide of fine granulometry (0.125 mm) is first introduced. Next, a series of silicon carbide beds of different particle sizes: 2 ml of 0.125 mm, 7 ml of 0.5 mm and finally 1.19 mm up to the top of the reactor, are introduced.

The reactor is then connected to the test installation. The temperature of the catalyst is adjusted to a temperature of 305° C. measured at the “dehydration layer”.

The reactor is fed with a gaseous mixture of helium-krypton/water-glycerol. The helium-krypton gaseous mixture contains 4.92% of krypton that serves as internal standard. The water-glycerol mixture contains 30% by weight of glycerol.

The constitution of the mixture injected is as follows, expressed as a mole percentage: helium/krypton/O₂/water/glycerol: 50/2.6/3.4/40.6/3.4.

The rate of introduction of the feed mixture is such that the hourly space velocity (HSV) will be 2000 h⁻¹. This introduction rate is expressed in HSV, i.e. as a throughput of mixture relative to the volume of catalyst used.

The effluents are collected at the reactor outlet by an ice cold trap for separating the liquid effluents from the incondensable matter. The acrolein, hydroxy-propanone and acrylic acid are assayed by chromatographic analysis.

The effluents are cumulated in the trap over a period of 60 minutes. The uncondensable gases are analyzed throughout the reaction balance. The yield of acrolein produced is 70 mol %, the acrylic acid content of the effluent is 2 mol % and the hydroxyacetone content is 0.5 mol %.

### Example 2

### Preparation of Acrolein Polymer

100 g of acrolein obtained in Example 1 stabilized with hydroquinone and diluted in 1 liter of demineralized water, at a room temperature close to 20° C., are introduced into a glass reactor comprising a mechanical stirrer. Aqueous 0.2M sodium hydroxide solution is then added so as to bring the acrolein solution to a pH of about 10.5. Almost immediately, the formation of a white precipitate is observed. After 30 minutes, the stirring is stopped and the solid product is recovered by filtration and then washed with water. After drying at room temperature for 2 days on filter paper, 42 g of solid polymer are recovered.

1. An acrolein-based polymer comprising units of the type: 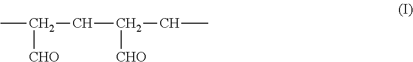
which may be in hydrated, hemiacetal or acetal form, characterized in that it has a mass content of ¹⁴C such that the ¹⁴C/¹²C ratio is between 0.2×10⁻¹² and 1.2×10⁻¹² according to standard ASTM D 6866.

2. The polymer as claimed in claim 1, characterized in that the units (I) result from the homopolymerization of acrolein or from the copolymerization of acrolein with at least one polymerizable comonomer.

3. The polymer as claimed in claim 2, characterized in that the comonomer is chosen from n-substituted acroleins such as aryl, arylalkyl, alkyl and alkylaryl acroleins, aldehyde derivatives of acrolein, monomers containing at least one ethylenic group, preferably a group CH₂═CH—, such as butadiene, isoprene, methylpentadiene, cyclopentadiene, chloroprene, ethylene, propylene, butylene, octene, vinyl acetate, vinyl propionate, vinylpyridine, vinylnaphthalene, styrene, vinylcyclohexane, vinyl chloride, vinylidene chloride, acrylic derivatives such as acrylic acid, methacrylic acid, acrylonitrile, methacrylonitrile, esters such as methyl methacrylate, ethyl acrylate, butyl acrylate, allylic compounds, such as allyl acetate, allyl alcohol, allylamine, diallyl succinate.

4. A process for preparing an acrolein-based polymer comprising units of the type: 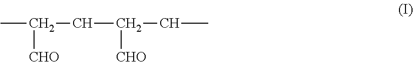
which may be in hydrated, hemiacetal or acetal form, as defined according to claim 1, comprising the following steps:
(a) the dehydration of glycerol starting with an aqueous solution of glycerol in the presence of an acid catalyst, to produce a mixture of acrolein and water in a first reactor;
(b) the optional purification of the product obtained in step (a);
(c) the polymerization in a second reactor of the acrolein produced in (a) or (b);
(d) the recovery of the polymer produced in (c), optionally followed by steps of washing, drying and/or molecular distillation and activation in air.

5. The process as claimed in claim 4, characterized in that step (b) comprises a partial condensation of water and of the heavy byproducts.

6. The process as claimed in claim 4, characterized in that the polymerization in the second reactor is performed in the presence of at least one comonomer that is polymerizable with acrolein, such as acrylic acid.

7. The process as claimed in claim 4, characterized in that the polymerization reaction is performed in aqueous solution in the presence of a base, and in the presence of air and/or oxygen.

8. The process as claimed in claim 4, characterized in that it comprises an additional step (e) that consists in heating the polymer recovered in step (d), in dry form, at a temperature ranging from room temperature to 110° C. and preferably from 60° C. to 85° C. in the presence of air to at least partially oxidize the aldehyde functions of said polymer.

9. The process as claimed in claim 4, characterized in that it comprises an additional step (e) that consists in heating the polymer in the presence of water, optionally in the presence of a polyethylene glycol, a polyol or an alkanol, at a temperature ranging from 40° C. to 150° C. and preferably from 40° C. to 115° C., for a time ranging from 1 to 1400 hours and preferably from 10 to 60 hours.

10. The process as claimed in claim 4, characterized in that it comprises an additional step (e) that consists in dissolving the polymer recovered in step (d) in an alcohol or a polyol, optionally in the presence of water, to form a solution with a pH of less than or equal to 7, in heating the solution thus obtained to form a product of reaction between said polymer and the alcohol or the polyol.

11. The process as claimed in claim 4, characterized in that it comprises an additional step (e) that consists in dissolving the polymer recovered in step (d), in a basic aqueous solution such as a sodium carbonate solution.

12. A composition comprising at least one polymer as claimed in claim 1, and a linear or branched C₁ to C₁₀ alcohol or a basic aqueous solution.

13. The composition as claimed in claim 12, in the form of a solution or in the form of an emulsion.

14. In medicaments intended for treating or preventing infections, food additives for cattle, dermatological compositions, a preserving agent or biocidal agent in drilling muds, paints, plastics, inks, paper or textiles comprising a polymer, the improvement wherein the polymer is one according to claim 1.
